(19) United States
(12) Patent Application Publication
(10) Pub. No.: US 2023/0146924 A1
(43) Pub. Date: May 11, 2023

(54) NEURAL NETWORK ANALYSIS OF LFA TEST STRIPS

(71) Applicant: EXA Health, INC., Los Altos, CA (US)

(72) Inventors: Mayank Kumar, Sunnyvale, CA (US); Kevin J. Miller, Mountain View, CA (US); Steven Scherf, Oakland, CA (US); Siddarth Satish, Redwood City, CA (US)

(21) Appl. No.: 17/421,841

(22) PCT Filed: Jul. 7, 2021
(86) PCT No.: PCT/US2021/040665

§ 371 Date: Jul. 9, 2021

Related U.S. Application Data
(60) Provisional application No. 63/049,213, filed on Jul. 8, 2020.

(51) Int. Cl. G06V 10/774; G01N 21/78; G16H 30/40; G16H 10/40; G06V 10/82; G06V 10/56; G06V 10/60

(52) U.S. Cl. CPC G06V 10/774; G01N 21/78; G16H 30/40; G16H 10/40; G06V 10/82; G06V 10/56; G06V 10/60; G01N 2021/7759

(57) ABSTRACT

Example methods and systems train an end-to-end neural network machine to analyze images of lateral flow assay test strips by learning non-linear interactions among lighting variations, test strip reflections, bi-directional reflectance distribution functions, angles of imaging, response curves of smartphone cameras, or any suitable combination thereof. Such example methods and systems improve the limit of detection, the limit of quantification, and the coefficient of variation in the precision of quantitative test results, under ambient light settings.

### RELATED APPLICATION

This application is a national phase entry of International Application No. PCT/US21/40665, titled “NEURAL NETWORK ANALYSIS OF LFA TEST STRIPS” and filed Jul. 7, 2021, which claims the priority benefit of U.S. Provisional Patent Application No. 63/049,213, titled “NEURAL NETWORK ANALYSIS OF LFA TEST STRIPS” and filed Jul. 8, 2020, which is incorporated herein by reference in its entirety.

### TECHNICAL FIELD

The subject matter disclosed herein generally relates to the technical field of special-purpose machines that facilitate analysis of test strips, including software-configured computerized variants of such special-purpose machines and improvements to such variants, and to the technologies by which such special-purpose machines become improved compared to other special-purpose machines that facilitate analysis of test strips. Specifically, the present disclosure addresses systems and methods to facilitate neural network analysis of test strips.

### BACKGROUND

Lateral Flow Assay (LFA) is a type of paper-based platform used to detect the concentration of analyte in a liquid sample. LFA test strips are cost-effective, simple, rapid, and portable tests (e.g., contained within LFA testing devices) that have become popular in biomedicine, agriculture, food science, and environment science, and have attracted considerable interest for their potential to provide instantaneous diagnostic results directly to patients. LFA-based tests are widely used in hospitals, physicians' offices, and clinical laboratories for qualitative and quantitative detection of specific antigens and antibodies, as well as for products of gene amplification. LFA tests have widespread and growing applications (e.g., in pregnancy tests, malaria tests, tests for COVID-19 antibody tests, COVID-10 antigen tests, or drug tests) and are well-suited for point-of-care (POC) applications.

### BRIEF DESCRIPTION OF THE DRAWINGS

Some embodiments are illustrated by way of example and not limitation in the figures of the accompanying drawings.

FIG. 1 is a pair of graphs that show observed results comparing the performance of such an end-to-end neural network machine, according to some example embodiments, in directly predicting concentrations of an analyte, to other approaches.

FIG. 2 is a block diagram illustrating an architecture and constituent components of an end-to-end neural network machine or other system configured to perform analysis of LFA test strips, according to some example embodiments.

FIG. 3 is a flow chart illustrating a method of identifying or otherwise determining (e.g., localizing) a portion of an image that depicts an LFA test device (e.g., an LFA test cassette), where the identified portion of the image depicts the LFA test strip of the LFA test device, according to some example embodiments.

FIGS. 4 and 5 are flow charts illustrating a method of training a neural network analysis to analyze LFA test strips, according to some example embodiments.

FIG. 6 is a block diagram illustrating components of a machine, according to some example embodiments, able to read instructions from a machine-readable medium and perform any one or more of the methodologies discussed herein.

### DETAILED DESCRIPTION

Example methods (e.g., algorithms) facilitate neural network analysis of test strips (e.g., LFA test strips), and example systems (e.g., special-purpose machines configured by special-purpose software) are configured to facilitate neural network analysis of test strips. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

LFA test strips usually have a designated control line region and a test line region. Typically, results can be interpreted within 5-30 minutes after putting a sample within the designated sample well of the LFA test device. The LFA test device may take the example form of an LFA test cassette, and the LFA test device typically has at least one sample well for receiving the sample to be applied to an LFA test strip inside the LFA test device. The results can be read by a trained healthcare practitioner (HCP) in a qualitative manner, such as by visually determining the presence or absence of a test result line appearing on the LFA test strip.

However, qualitative assessment by a human HCP may be subjective and error prone, particularly for faint lines that are difficult to visually identify. Instead, quantitative assessment of line presence or absence, such as by measuring line intensity or other indicator of line strength, may be more desirable for accurate reading of faint test result lines. Fully or partially quantitative approaches directly quantify the intensity or strength of the test result line or can potentially determine the concentration of the analyte in the sample based on the quantified intensity or other quantified strength of the test result line. Dedicated hardware devices to acquire images of LFA test strips and image processing software to perform colorimetric analysis to determine line strength often rely upon control of dedicated illumination, blockage of external lighting, and expansive equipment and software to function properly. More flexible and less expensive approaches may be beneficial.

The methods and systems discussed herein describe a technology for smartphone-based LFA reading and analysis. The technology utilizes computer vision and machine learning (e.g., a deep-learning neural network) to enable a suitably programmed smartphone or other mobile computing device to function as a high-end laboratory-grade LFA reader configured to perform qualitative measurements, quantitative measurements, or both, on a wide variety of LFA test strips under a wide variety of ambient lighting conditions. Specifically, the methods and systems discussed herein are not reliant upon controlling dedicated light sources or use of light-blocking enclosures for accurate interpretation of LFA test results. The methods and systems disclosed herein can be used to train a neural network to interpret LFA test results for a variety of applications, such as malaria tests, COVID-19 antibody tests, COVID-19 antigen tests, cancer tests, and the like, and can be adapted to work with any number of different makes, models, or other types of LFA test devices (e.g., various LFA test cassettes) that house LFA test strips.

Existing methods and systems that use a smartphone camera under ambient light settings to fully or partially perform quantitative assessment of LFA test strips generally do so by performing linear colorimetric light normalization on images captured by the smartphone camera. However, such linear colorimetric light normalization (e.g., by dividing the intensity of a test result line by the intensity of a control line, or by dividing the intensity of the test line by background color of the test strip) may yield inaccurate results in practical settings, such as where the interaction of ambient light and the test strip are complicated by the angle of imaging, multiple light sources of varying color temperature, shadows, glare, specular reflections, and non-linear response-curves of consumer-grade cameras (e.g. power-law gamma-response curves of smartphone cameras).

In contrast, the methods and systems discussed herein train an end-to-end neural network machine to learn such non-linear and complicated interactions among lighting variations, test strip reflections (e.g., albedo), bi-directional reflectance distribution functions (BRDF) of test strips, angles of imaging, response curves of smartphone cameras, or any suitable combination thereof. The methods and systems accordingly improve the limit of detection (LOD), the limit of quantification (LOQ), and the coefficient of variation (COV) (e.g., representing the precision of quantitative test result interpretation, analyte concentration predictions, or both), under ambient light settings.

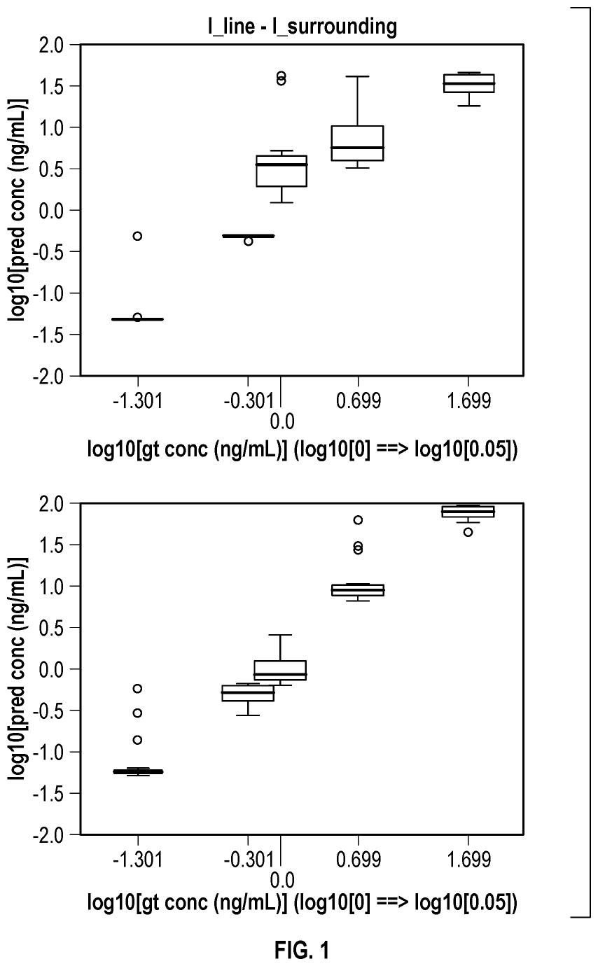

FIG. 1 is a pair of graphs that show observed results from such an end-to-end neural network machine, according to some example embodiments, in directly predicting concentrations of an analyte, in comparison to other approaches that use line intensity features and linear light normalization with regression analysis. Specifically, the upper graph of FIG. 1 depicts the performance of approaches based on colorimetric analysis and light normalization, while the lower graph of FIG. 1 depicts the performance of an example embodiment of an end-to-end neural network machine, under varying ambient light conditions, with changes in color temperature and angle of imaging.

As shown in the upper graph shown in FIG. 1, approaches based on colorimetric analysis and light normalization result in higher variance and higher COV. As seen in the lower graph shown in FIG. 1, the trained end-to-end neural network machine performed better, with lower variance and better COV, and therefore obtained improvements in LOD and LOQ for quantitative assessment of LFA test strips.

Training a neural network machine for accurate performance usually uses a large amount of labeled training examples, such as many examples of test strip images with varying levels of strength (e.g., intensity) for test result lines and control lines. For example, the training database may contain a training set of images that depict LFA test strips with a mixture of strong lines, weak lines, faint lines, no lines, etc., for both test result lines and control lines, as well as ground truth qualitative labels (e.g., indicating presence or absence of a line), ground truth quantitative labels (e.g., indicating concentration of analyte), or both. Moreover, the training images may also vary their respective imaging conditions, such as lighting conditions (e.g., color, intensity, and shading), exposure, imaging angles, imaging locations, test strip backgrounds (e.g., with varying amounts of stains from samples, blood, or both), to generate a representative training dataset that can be used to train a neural network machine to perform qualitative and quantitative assessment of LFA test strips in practical settings. However, collecting and labelling such a large amount of training data may be prohibitively expensive, time consuming, or both.

According to the methods and systems discussed herein, realistic looking images of LFA test strips for training a neural network machine may be simulated with widely varying parameters. Examples of such parameters include line strength (e.g., with a line strength parameter that can range from 0 (no line) to 1 (strong line)), line color, line thickness, line location, or any suitable combination thereof. Other examples of such parameters indicate variations in test strip background (e.g., with or without blood stains), lighting conditions, shadows, or any suitable combination thereof. These simulated test strip images can be generated by a suitable machine (e.g., by a module or other feature programmed into the neural network machine) and then used to fully or partially train (e.g., pre-train) the neural network machine. Such simulated images are particularly effective in helping the neural network machine detect and appropriately quantify the faintest of test result lines, as well as render these machine-made inferences less sensitive to lighting conditions or other imaging conditions, as well as more sensitive to the line strength parameters used in generating these simulated images.

In some example embodiments of the methods and systems discussed herein, a neural network machine is pre-trained on generated simulated images of LFA test strips and then fine-tuned (e.g., via further training) for a specific application domain, such as performing assessment of images of actual LFA test strips with a limited amount of data. Such assessment may include qualitive assessment (e.g., presence or absence of test result lines), direct quantitative assessment of test results, or any suitable combination thereof. The fine-tuning of the neural network machine enables the neural network machine to directly predict the presence or absence of a test result line for a particular application, predict the concentration of the analyte in some other application, or both. According to some example embodiments, the methods and systems disclosed herein can help train a neural network machine to detect faint test result lines that indicate positive or negative results for COVID-19 antibody tests, COVID-19 antigen tests, or both, without first obtaining a large training set of labeled images showing positive or negative COVID-19 antibody test strips, COVID-19 antigen test strips, or both. Therefore, pre-training the neural network machine with generated photo-realistic simulated images, coupled with further training for a specific downstream task, may reduce or avoid the cost, effort, or resource usage involved in obtaining large amounts of labeled images of actual test strips.

Certain example embodiments of the methods and systems discussed herein include use of a modified camera (e.g., a modified smartphone camera or other modified camera hardware), modified image acquisition, or both, to further improve the sensitivity of the trained neural network machine in detecting faint lines (e.g., faint test result lines), as well as to perform accurate quantitative assessment of LFA test strip images. In particular, such modifications to hardware, image acquisition, or both, may include acquiring an image of a test device (e.g., a test cassette) with and without flash illumination, acquiring multiple images under varying exposure to increase the dynamic range of the camera, acquiring images in RAW imaging format to avoid artifacts from image processing, programmatically adjusting one or more camera parameters (e.g., image sensor sensitivity (“ISO”), exposure, gain, white balance, or focus), or any suitable combination thereof, to optimize the performance of the trained neural network machine in assessing (e.g., interpreting) images of LFA test result lines.

To perform automated LFA analysis using a smartphone with a camera, an image of an LFA test device (e.g., an LFA test cassette) is acquired. The image may be acquired using a specific image acquisition methodology that optimizes the detection of faint lines (e.g., faint test result lines appearing on an LFA test strip included in the LFA test device) and the linearity of the camera's response. Once the image is acquired, the first step in the image analysis process is to localize the region of the image that shows the test device, the corresponding sub-region that shows the result well of the test device, or both. In some example embodiments, a separate (e.g., secondary) neural network machine is configured and trained to detect a specific type of test device appearing within an image. Whether separate or not, such a neural network machine can be trained to recognize any of several types (e.g., makes, models, etc.) of test devices. In other example embodiments, such a neural network machine is configured to recognize just one unique type of test device. Once the portion (e.g., sub-region) of the image that depicts the result well of the test device is identified (e.g., by location coordinates of the image portion within the image), one or more further refinements of the location coordinates of the test strip (e.g., a visible portion of the test strip, as that portion appears in the result well) may be performed to accurately identify and crop-out just the sub-sub-region of the image showing only the test strip or portion thereof, for further LFA analysis. The second step performs the actual analysis of the cropped portion of the image (e.g., the cropped sub-sub-region that shows the test strip or portion thereof). This analysis may be performed by an end-to-end neural network machine trained to perform qualitative assessments, quantitative assessments, or both, of the portion of the image. The end-to-end neural network machine may have been pre-trained on generated simulated images of LFA test strips and then fine-tuned for a specific application.

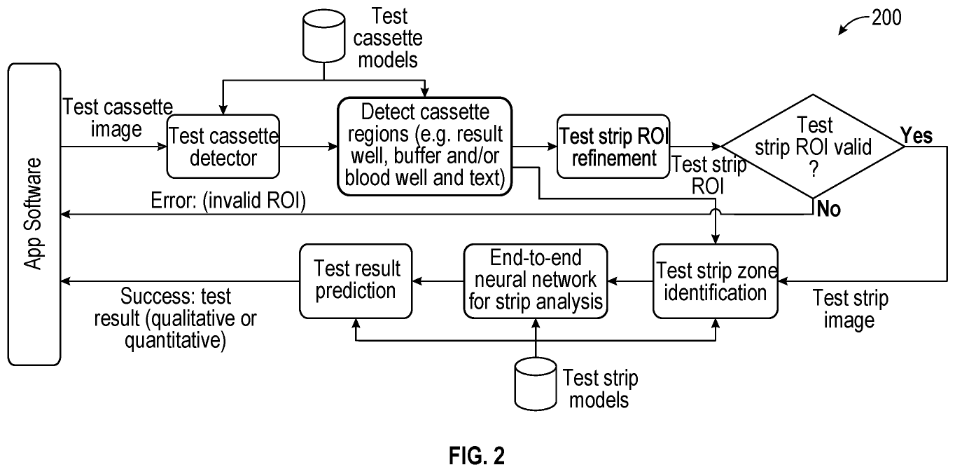

FIG. 2 is a block diagram illustrating an architecture 200 and constituent components of an end-to-end neural network machine or other system configured to perform analysis of LFA test strips, according to some example embodiments.

In some example embodiments of the image acquisition methodology, images of test devices (e.g., images depicting LFA test cassette) are acquired using an application that executes on smartphones and captures such images with the smartphones' flash turned on (e.g., set to an ON state). Capturing images with flash illumination may be termed “flash imaging” and may be performed to avoid shadows directly falling on the test strip region of an image (e.g., the sub-sub-region that depicts the test strip or a portion thereof), to reduce the amount of light for accurately detecting one or more test results lines on the test strip, or both.

In certain example embodiments of the image acquisition methodology, two test device images are acquired —one with flash turned on, and the other with flash turned off (e.g., set to an OFF state), and thereafter a delta image showing the differences between these two images is generated via software by subtracting one image from the other (e.g., I_{Flash}-I_{NoFlash}). This approach minimizes or removes the effects of ambient lighting on the resulting test device image (e.g., the delta image). That is, in the resulting delta image, which may be called a “difference image,” external light sources will have minimum to no impact on the appearance of the test device. This approach can be helpful to avoid using any full or partial enclosures (e.g., cardboard or fiberboard enclosures or other dedicated light-blocking hardware) to reduce the amount of ambient light reaching a test device to be imaged.

In other example embodiments of the image acquisition methodology, multiple (e.g., several) images of a test device are captured, each with a different level of exposure, a different ISO, or a different combination thereof, for high dynamic range (HDR) imaging. The combined HDR images have a much higher sensitivity and can be used to detect fainter lines than when just using a single image. As a result, HDR imaging increases the limit of detectability for faint test result lines.

In addition, the acquired images of LFA test devices may be stored in a lossless manner (e.g., as Portable Network Graphics (PNG) images), such that compression artifacts do not adversely affect image quality or remove small signals that indicate faint lines from the stored images. In some example embodiments, unprocessed or minimally processed raw (“RAW”) images (e.g., containing raw data from the camera's optical sensor) can be used for performing test strip analysis. Such RAW images are possible to acquire using contemporary generations of smartphones and may be beneficial to use for LFA analysis, at least because the response curve for the camera is more linear in RAW images than in gamma-corrected images or other post-processed images. Also, RAW images may provide a higher number of bits per pixel compared to Joint Photographic Experts Group (JPEG) images, thereby increasing the limit of detectability for the same camera hardware within a given smartphone.

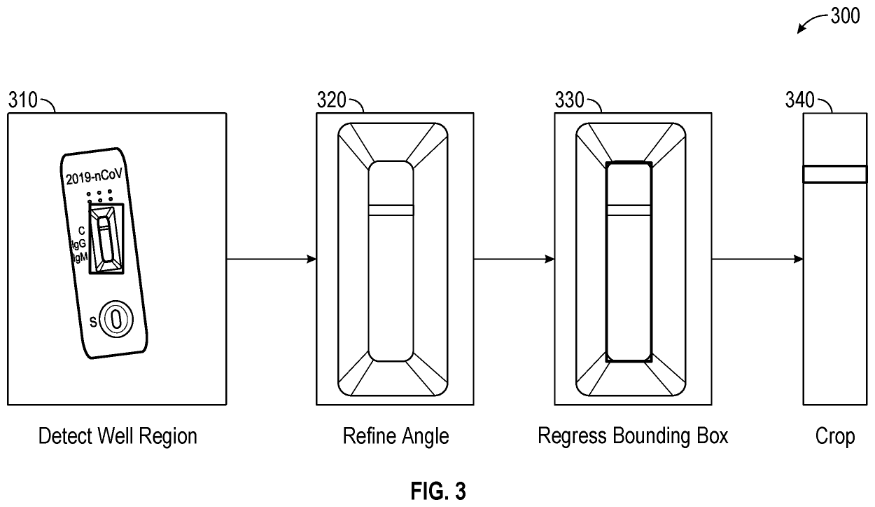

FIG. 3 is a flow chart illustrating a method 300 of identifying or otherwise determining (e.g., localizing) a portion of an image that depicts an LFA test device (e.g., an LFA test cassette), where the identified portion of the image depicts the LFA test strip of the LFA test device. For example, the portion of the image may be identified by identifying or otherwise determining a region of the image, where the region depicts the LFA test device, then identifying (operation 310) or otherwise determining a sub-region of the region, where the sub-region depicts the result well of the LFA test device, then aligning (operation 320) the sub-region for further processing, and then identifying (operation 330) or otherwise determining a sub-sub-region of the sub-region, where the sub-sub-region depicts the LFA test strip or portion thereof, visible in the result well of the LFA test device, before cropping (operation 340) the sub-sub-region that depicts the LFA test strip.

As shown in FIG. 3, according to some example embodiments of the method 300, the operations (e.g., operations 310, 320, 330, and 340) of the method 300 are performed to identify or otherwise determine (e.g., localize) and then crop out the test strip sub-sub-region for analysis by a neural network machine. The identifying of the region that shows the test device may include using one or more object-detection models for neural networks (e.g., object-detection models named Yolo, SSD, Faster-RCNN, Mask-RCNN, CenterNet, or any suitable combination thereof) to predict or otherwise determine a bounding box (e.g., an upright rectangular bounding box with portrait orientation) around the entire result well, as depicted in the image, and crop that portion of the image to obtain the sub-region that shows the result well of the test device.

For further refinement of the coordinates for the sub-region that shows the result well, the method 300 may include extracting one or more edge maps from this cropped sub-region and remove any small connected components from the extracted edge maps, as those tend to be noisy, irrelevant, or both. The method 300 may then also include applying a Hough transform on such an edge map, discarding any Hough lines whose orientation is more horizontal than vertical, clustering the Hough lines to consolidate any duplicate lines, removing any Hough lines whose orientation is too far from the median (e.g., beyond a threshold deviation from the median), and averaging the orientations of the remaining Hough lines. The resulting averaged orientation (e.g., used as an estimate) may thus be a basis for rotating the sub-region that shows the result well, such that the sub-sub-region that shows the test strip or portion thereof will be upright or as close to upright as possible. In certain example embodiments, a separate (e.g., second) neural network machine (e.g., a second convolutional neural network machine) determines regresses a tight bounding box (e.g., via regression) around the result well, as depicted in this upright sub-region of the image.

In some example embodiments, a variant of the CenterNet object detection model is used in the identifying of the region that shows the test device, within the image of the test device. The variant of the CenterNet object detection model can detect rotated bounding boxes directly and can thus be trained to localize the sub-sub-region that shows the test strip or portion thereof. This approach has fewer steps than using other object detection models, but this approach also relies on more manual labelling effort, since each labelling involves at least three points (e.g., the width and height of an upright bounding box, and its rotation angle), instead of just two points.

In certain example embodiments, another variant of the CenterNet object detection model acts as a keypoint detection model and can detect any arbitrary four coordinates to localize the sub-region in which the result well appears in the image of the LFA test device. This approach easily handles rotations that are outside of the camera plane, since a homography transform can be used to warp the quadrilateral region into an upright rectangular shape. However, this approach involves four points per manual label.

In most typical use cases, the sub-region in which the result well appears in the image of the LFA test device (e.g., with the sub-region being called the “result well region”), the sub-sub-region in which the test strip or portion thereof appear (e.g., with the sub-sub-region being called the “test strip region”), or both, are visually distinctive enough for the above-discussed methodologies to perform accurately. However, in situations where this may not be the case, one or more object detection models may be configured to detect one or more other landmark features on the test device (e.g., the test cassette), such as text or markings (e.g., circles) on the housing of the test device, and use the known geometry of the test device to infer the location of the corners of the test strip region. This inference may be performed using a homography transform, as the test strip region is generally almost coplanar with the front of the test device. The technique of determining a tight bounding-box—(e.g., via regression) using a convolutional neural network (e.g., as discussed above) can be used to refine this inference (e.g., as an initial estimate), to account for any errors due to the test strip being slightly out of plane with the front of the test device.

In various example embodiments, the object detection model directly detects the result well region, without previously detecting the whole test device within the image. Such example embodiments may be advantageous in situations where multiple brands of test devices have similar looking result well regions, or where the images to be analyzed show only the result well (e.g., due to original image capture or due to prior cropping), without showing the entire test device. In some implementations, a neural network machine can be trained on just one or two brands of test devices, and the resulting trained neural network machine can successfully detect the result well regions of images depicting other brands of test devices, where the test devices of these other brands are not depicted in the training set of images. This capability thereby reduces the size of the training dataset and its expense for acquisition. The same principle may be applied to omit one or more other operations (e.g., operation 320) in the method 300 discussed above. However, in situations where different brands of test devices do not have similar looking result well regions, one or more additional landmarks can be used to localize the test strip region of the images. Specifically, a neural network machine can trained to find landmarks that appear in both the training brands and the brands to be tested, to detect these landmarks, and to use the known geometry of a particular brand of test devices to infer the location of the test strip region for that particular brand of test devices. Examples of such landmarks include the corners of the test device, the corners of the sample well on the test device, text appearing on the test device (e.g. “C,” “G,” “M,” “T,” “IgG,” “IgM,” “COVID-19,” “SARS-Cov-2,” “Pv,” “Pf,” or any suitable combination thereof), one or more holes in the housing of the test device, one or more markings (e.g., lines or ridges), or any suitable combination thereof.

Once the test strip region is accurately localized, the next task in the analysis is to estimate the line strength (e.g., line intensity) of the test result line, with or without estimating the line strength of the control line, to perform automated assessment (e.g., qualitative, quantitative, or both) of the test strip. As noted above, an end-to-end trainable neural network machine is trained to either directly predict the presence or absence of a test result line (e.g., for qualitative assessment) or perform analyte concentration prediction (e.g., for quantitative assessment). The neural network machine may be configured to easily learn the non-linear and complicated interactions among lights, test strip reflection (e.g., albedo), other optical effects (e.g., shadows), or any suitable combination thereof. The trained neural network machine takes, as input, the image of the test strip or a cropped test strip region. Additionally, the neural network machine may also take, as input, one or more parameters indicative of auxiliary lighting, one or more other imaging conditions, or any suitable combination thereof. The output of the trained neural network machine may thus include a directly determined probability of the presence or absence of the test result line, the strength of the test result line or its underlying concentration of the analyte. The neural network machine may be further trained with images of the test device under various imaging conditions, such as ranges for light intensity, color temperature, imaging angle, or any suitable combination thereof, such the neural network machine learns to disregard or nullify such variations in imaging conditions while performing the above-described assessments (e.g., qualitative, quantitative, or both). Generally, the more data collected and used in the training phase, the better the resulting accuracy in the trained neural network machine. Thus, the training data can be augmented with artificially simulated variations depicted in generated training images, where such variations include changing the brightness, contrast, hue, saturation, or any suitable combination thereof, in the images to mimic realistic variations and increase the amount of training data.

In some example embodiments, the neural network machine implements an object detection model that includes two parts, A and B. Part A is a fully convolutional neural network. Its input is an image of the test strip region (e.g., cropped as described above), and its output is a three-dimensional (3D) array of activations. This output may be converted to a one-dimensional (1D) vector, using a global spatial average (e.g., Global Average Pooling), which can be a uniform average or a weighted average with learnable weights.

Alternatively, the 1D vector may be obtained by flattening the 3D array. This 1D vector may then be fed into Part B, which is a neural network with dense connections. Part B outputs predictions of the target variables, such as line presence probability (e.g., for qualitative assessment), line strength alpha (e.g., for semi-quantitative or partially quantitative assessment), analyte concentration (e.g., for quantitative assessment), or any suitable combination thereof. Part B may also output predictions of one or more other variables that can be used to supervise the training of the neural network, such as the location of the test result line within the input image of the test strip region, the locations of the top and bottom of the test result line, or both. If a test device has multiple test result lines (e.g. C-G-M test result lines in a COVID-19 test device, C-IgG-IgM test result lines, or C-T test result lines), then the test strip region may be divided into smaller regions to be independently analyzed in a manner similar to methodologies discussed herein for a single test result line. In alternative example embodiments, neural network machine is trained to analyze the entire test strip region (e.g., as a whole) and produce multiple outputs, one for each of the test result lines.

In certain example embodiments, the neural network machine implements deep learning fused with signal processing. In this approach, Part A of the object detection model is still a fully convolutional network, but it outputs a single two-dimensional (2D) heatmap, which is averaged across the horizontal dimension (e.g., parallel to the one or more test result lines) to give a 1D profile similar to a ID intensity profile. Then, Part B is a peak detection algorithm whose loss function is differentiable with respect to the 1D profile. As an example, suppose Part B found the highest intensity of the 1D profile and compared the highest intensity to a threshold intensity (e.g., a predetermined threshold value for intensity) to decide whether a test result line is present. Then, Part B can convert each profile intensity z to a probability p using the logistic regression equation p=1/(1+exp(−az+b)), where a and b are learnable, and then impose a binary cross entropy loss on the probabilities, encouraging the probability to be near 1 at the center of the line and be 0 at all locations that are not very close to the line. As another example, suppose Part B measured the topographic prominence (e.g., the autonomous height, the relative height, or the shoulder drop) of each entry in the profile, but Part B is modified to compute the minimums within a fixed-size window. Then, the topographic prominences would be a differentiable function of the intensity profile, and a logistic layer and a binary cross entropy loss could be used. If Part B is differentiable, then the entire object detection model can be trained end-to-end. A reconstruction loss can be used to encourage Part A to generate an image that is similar to its input, while the loss from Part B should encourage Part B to generate an image that is more conducive to peak detection. Part A can be architected in a way that limits the type of changes Part A can make to the image (e.g., limit the receptive field of each output activation). This approach might be less prone to overfitting than other approaches.

To make the object detection model more robust to ambient lighting variations, the object detection model may be given the color of a “reference” part of a test device (e.g., as an extra input or an auxiliary input), such that the object detection model can use this information to learn the non-linear effects of lighting and imaging angles for the specific test strip or test device, during the training phase. This “reference” part may be a portion of the test device that is outside the result well and free from text, as such a portion would have a constant color and would have the same orientation and incident angle as the test strip. Alternatively, the “reference” portion could be a blank part of the test strip itself (e.g., having no line whatsoever). Each of these options has advantages and disadvantages. The region outside of the result well may be made of a different material than the test strip, and it might have a different BRDF, but the region may not suffer from fluid gradients, which may be especially pronounced if the sample fluid is blood. If the region outside the result well is used, then it may be helpful to avoid glare or other strong instances of specular light. This can be done using noise-removal techniques such as Maximally Stable Extremal Regions (MSER), Otsu thresholding, median thresholding, or any suitable combination thereof. Alternatively, two images of the same test device (e.g., test cassette) under different imaging angles may be captures and then warped into alignment via keypoint matching, followed by taking a pixel-wise minimum to eliminate any specular highlights (e.g., specularities).

There are several ways to use the “reference” color to learn the lighting dependent variations in appearance of LFA test strips. One technique is to feed both the test strip image and the reference color into a neural network (e.g., in the neural network machine), and train the neural network on how to normalize and correct for lighting variations during the training phase. Because the reference color is a vector, the reference color can be concatenated onto the input of the densely connected part of the neural network. The reference color can also be concatenated onto the input for any intermediate layer or output layer of the densely connected part of the neural network. Furthermore, the reference color can be broadcast into a 3D array and concatenated into the input for any convolutional layer. The reference color can be fed through one or more of the dense layers before being broadcast or concatenated.

According to some example embodiments of the methods and systems discussed herein, the training of the neural network machine includes supervision to teach the neural network how to use the reference color to normalize an image of an LFA test strip. For example, the neural network may be configured to output an image, and training process may penalize differences between this output image and a reference image that was normalized by a reference technique (e.g. by dividing image colors by a reference color). The weight of this penalty may be reduced over time during the training process, such that the neural network learns an improved normalization technique of its own. As a result, the learned normalization technique models the non-linear interactions that are usually seen in such settings due to variations in lighting, imaging angle, camera response curve, etc. In certain example embodiments, this output is a separate head at the end of the neural network. In other example embodiments, the convolutional part of the neural network is split into two parts, such as a lower part that generates a normalized image, and an upper part that infers the presence or absence of lines in that normalized image. In either case, the extra supervision may prevent the extra input of reference color from causing overfitting.

For best results, a large amount of training data should be used when training a neural network. This is even more important when the neural network is being trained to account for variations in lighting, imaging conditions, etc., as described above. Accordingly, in various example embodiments of the methods and systems discussed herein, one or more methodologies for generating simulated photo-realistic images of LFA test strips are included in the training process or preparations therefor. That is, in various example embodiments, a machine (e.g., a machine configured to train or become the neural network machine) programmatically generates a large number of simulated test strip images with varying simulation parameters.

To synthesize an image of an LFA test strip (e.g., in an LFA test device), the machine performing the image synthesis obtains an image (e.g., a first image) of a test result line and an image (e.g., a second image) of a blank test strip background, and the machine then combines the two images to generate one or more artificial images of test strips. In generating the artificial images, the machine varies the color and brightness in the background image (e.g., the second image) to simulate lighting variations, artificially adds color smears to simulate patterns of fluid (e.g., blood) that may often appear on real LFA test strips, or both. In similar fashion, the machine may vary the foreground image (e.g., the first image) with respect to test result line, such as its color, thickness, orientation, location, or any suitable combination thereof. In addition, the machine may vary one or more alpha blending parameters (e.g., within a range of alpha values, such as between 0 (no line) to 1 (strong line)) to simulate test result lines of varying strength, as are often seen in actual images of LFA test strips or portions thereof.

In some example embodiments of the image synthesizing process, the machine performing the image synthesis accesses (e.g., reads, requests, or retrieves) a set of images that depict LFA test strips (e.g., cropped from larger images of LFA test devices, as discussed above) and are known to exhibit strong test result lines. The machine may implement Otsu thresholding to create a rough bounding-box around each strong test result line. The rough bounding box may be manually refined and labeled as strong or faint (e.g., by a human operator of the machine). The machine performing the image synthesis may then use these bounding-boxes to initialize segmentation of the test result line (e.g., using GrabCut or a similar technique), to obtain a precise segmentation of the pixels that constitute the test result line. The resulting segmentation (e.g., treated as a further cropped portion of the input image) becomes a basis for generating realistic synthesized images of test result lines.

In certain example embodiments of the image synthesizing process, the machine performing the image synthesis directly simulates a test result line based on realistic parameters for color, thickness, etc. To obtain suitable background images, the machine may access a set of images of unused LFA test strips and then sample portions of the images that are known to have no test result lines (e.g. between test result lines or below the control line if the test strip is showing a negative test result). Alternatively, the machine may perform one or more painting operations to digitally remove any visible lines, which may be performed after first using one or more line segmentation masks to mark the area to be painted.

To combine a foreground test result line with a background, the machine performing the image synthesis may alpha-blend the image of the test result line onto a part of the background image, which may be done using the following equation:

I_{synth}(X+a,y+b)=alpha*Iₗᵢₙₑ(x,y)+(1−alpha)*I_{background}(x+a,y+b)

In this equation, alpha is the line strength to be simulated, and a and b are offsets specifying where to draw the line onto the background. The value of alpha falls into a range between 0 and 1, where a value of 1 would simulate a full-strength line, and a value close to 0 would simulate an extremely faint line. The value of a would typically be 0, and the value of b would be sampled from a distribution that reflects the range of vertical locations where a line is expected to be found. Iₗᵢₙₑ can be slightly rotated to simulate small errors in orientation from the detection of the LFA test strip, variations in orientation due the manufacturing process, or both. Both Iₗᵢₙₑ and I_{background} can be randomly flipped vertically and horizontally to create more variation.

However, in using line images segmented from pre-existing test strip images, the color of Iₗᵢₙₑ depends on both the strength of the source line and the lighting of the source image. This means that the line in I_{synth} could appear stronger or fainter for a fixed alpha, due to the source image being captured in a brighter or dimmer ambient lighting environment. In the worst case, Iₗᵢₙₑ could be the same color as I_{background} due to the former being captured in a very bright environment and the latter being captured in a very dark environment. To address this vulnerability, some example embodiments of the machine performing the image synthesis use the regions around the line to perform a normalized alpha-blend. For example, let I_{behind} be an estimate of what Iₗᵢₙₑ would look like if there was no line. I_{behind} can be created by taking the pixels in the source line image immediately above or below the Iₗᵢₙₑ pixels, by taking an average of the pixels both above and below, or by painting (e.g., inpainting) over the Iₗᵢₙₑ pixels. Then, the normalized alpha-blend equation becomes:

I_{synth}(x+a,y+b)=alpha*Iₗᵢₙₑ(x,y)I_{behind}(x,y)*I_{background}(X+a,y+b)+(1−alpha)*I_{background}(X+a,y+b)

This normalized alpha-blending may be especially useful in cases where the source line or the source background image has shadows, as the normalized alpha-blending removes the shadows from the source line image and incorporates the shadows from the source background image, such that the resulting synthetic image will have natural-looking shadows.

Normalized alpha-blending assumes that the source line will always be consistently strong. However, this assumption might not always hold true, even if the source line is sampled exclusively from control lines. As a result, simulated lines with the same alpha might appear to be fainter or darker, which may hinder the training of the neural network machine. To address this vulnerability, some example embodiments of the machine performing the image synthesis use only those simulated test line images that are known to be generated with similar intensity. Another approach to address this vulnerability is to assume that the concentration of the substance bound at the source lines is known, such as where the source lines are sampled from a previously labeled training set to seed the image synthesizing process. If the concentration is known, then an equation that expresses the Beer-Lambert law can be fitted to the source line data to compute a pre-existing alpha for each source line.

Suppose there is a Beer-Lambert function ƒ(conc) that describes the reflectance of the line for a given concentration conc of analyte and a given lighting L (x,y). In other words:

Iₗᵢₙₑ(x,y,conc)=L(x,y)*f(conc)

This function ƒ may be a short Beer-Lambert function ƒ(conc)=beta*exp(−gamma*conc), or the function ƒ may have one or more additional polynomial terms to account for any physical deviations from the ideal Beer-Lambert law. The function ƒ can be fitted to a dataset where L (x,y) is known. Alternatively, it could be assumed that I_{behind}(x,y)˜=Iₗᵢₙₑ(x,y,0), so f can be fitted to the relation:

Iₗᵢₙₑ(x,y,conc)/I_{behind}(x,y)˜=f(conc)/f(0)

Next, suppose that conc_{ref} is a concentration of analyte that is known to produce a line that looks strong. Then, the line color can be expressed as an alpha-blend of a conc_{ref} line and a zero-concentration (e.g., blank) line:

Iₗᵢₙₑ(x,y,conc)=alphaₚᵣₑ*L(x,y)*f(conc_{ref})+(1−alphaₚᵣₑ)*L(x,y)*f(0)

Accordingly,alphaₚᵣₑ can be expressed in terms of conc:

alphaₚᵣₑ=(f(conc)−f(0))/(f(conc_{ref})−f(0))

When simulated images are generated from source lines with known concentrations conc, the alpha-blending alpha can be compensated to account for alphaₚᵣₑ, such that the resulting generated set of synthetic images are more consistent, even when the synthetic images are derived from different images of the test strip, where the different images exhibit different source line strengths. This approach may be particularly useful in cases where some of the source line images have known concentrations, and the rest of the source line images have unknown concentrations but consistent line strength, due to the fact that this approach allows the combining of both sets of images. Also, this approach does not rely on the source background images having known concentrations of analyte.

In various example embodiments of the image synthesizing process, the machine performing the image synthesis simulates background images instead of relying on images of actual backgrounds. This approach may be useful when physical copies of the LFA test device are in short supply. Background variation can be caused by simulating variations in lighting, reflectance, debris, or any suitable combination thereof. Reflectance variations may be caused diffusion of liquids (e.g., unevenly) into the test strip (e.g., throughout one or more membranes of the test strip). Such variations in reflectance can be modeled using one or more modeling techniques. For example, for a blood-based LFA test strip, the heat equation can model the diffusion of blood into the test strip, and a Beer-Lambert equation can relate blood density to color. One or more models of capillary action can be implemented to account for the fact that the fluid is diffusing into a dry membrane. Additionally, physical samples of the LFA membrane material can be collected and infused with amounts of banked blood to create experimentally derived reference data for fitting these or other types of models. Given a large enough supply of membrane material and fluid (e.g., blood), one or more generative adversarial networks (GANs) may be trained to generate source backgrounds, instead of modelling the actual physics involved in the diffusion of such fluids.

To make the trained neural net machine more robust to lighting variations, it may be helpful to simulate lighting variations in the training data. In some example embodiments of the image synthesizing process, the machine performing the image synthesis applies one or more digital color augmentations to the simulated images. Examples of such augmentations include small shifts in brightness, color temperature, pixel values (e.g., in the hue-saturation-value (HSV) color space), or any suitable combination thereof. Other examples of augmentation include gamma correction and contrast adjustments, although augmenting these might make hamper the resulting trained neural network in accurately predicting alpha. To ensure that the simulated images still look realistic, the preparation of training data may include collecting images of any object that is same color as the test device in the target environment (e.g. a home, a clinic, or outdoors) and using color statistics to optimize augmentation parameters. This approach may be especially useful in cases where the source line images and the source background images cannot be collected in all possible target environments.

Shadows can provide a challenging source of variations in test strip images, because shadows generally are not spatially uniform. In some example embodiments of the image synthesizing process, the machine performing the image synthesis simulates one or more shadows by accessing (e.g., recovering or otherwise obtaining) a 3D structure (e.g., in the form of a 3D point cloud or other 3D model) of the test device and combining that structure with one or more simulated light sources. The 3D structure can be accessed, for example, using two different approaches. The first approach begins with acquiring a few images of the test device using different camera locations and camera angles and then finding 2D point correspondences for keypoints along the top and bottom walls of the result well of the test device. These correspondences can be labelled manually in instances where the keypoints do not lie on strong corners (e.g., at the apex of a rounded corner). The correspondences can then be used to recover the 3D coordinates of the keypoints, which in most cases would be enough information to create a 3D model of the test device (e.g., with most surfaces being flat, cylindrical, conical, or some suitable combination thereof). The second approach begins by imaging a test device under different lighting directions and then using a shape-from-shading technique to access (e.g., recover or obtain) the 3D structure of the test device. Once the 3D structure is accessed, one or more of various techniques can be used to simulate one or more shadows on the strip region. For example, if it is assumed that the light comes from a collection of point sources, then each pixel of the test strip image can be processed by computing how much light that pixel receives from each point source of light, based on the distance and angle to the point source and whether the point source is occluded by the 3D structure of the test device. For example, the machine performing the image synthesis may perform 3D rendering (e.g., ray tracing) of the scene to simulate shadows and directional lighting.

Debris can provide another challenging source of variations in test strip images. In particular, the presence of human hairs are a common source of error, because hairs can be easily overlooked by users. In certain example embodiments of the image synthesizing process, the machine performing the image synthesis simulates hairs by randomly drawing smooth, thin, dark-colored, curved lines onto the simulated images. Because hairs are generally very thin, hairs would not require much textural data to simulate with sufficient accuracy for purposes of training the neural network machine. Hairs can also be simulated in a more data-driven way, by imaging some actual hairs against a white background, segmenting the depicted hairs, and pasting the segmented hairs onto the simulated images of test strips or portions thereof. In both of these approaches, actual test devices with actual hairs on them need not be obtained. Similar approaches can be used to simulate other small, uniform-colored debris that might occur in non-laboratory settings.

In practical scenarios, it may be helpful to simulate as many types of variations as possible to generate simulated test strip images for training (e.g., pre-training) the neural network machine for automated LFA test strip analysis. In some example embodiments of the methods and systems discussed herein, pre-training with simulated images is later followed by fine-tuning the neural network with realistic training images. During the pre-training with simulated images, the neural network machine may be trained to predict more targets than during the later training with realistic images. This arrangement may be implemented on grounds that, with simulated images, more ground truth information is known than just the ground truth presence or absence of a test result line or the ground truth analyte concentration, than is usually available with non-simulated real training images. In certain example embodiments, the neural network machine is trained by configuring the neural network to predict not only the alpha, the analyte concentration, or both, but also to predict the line location, the line boundaries (e.g., y-coordinates of the top and bottom edges of the line), the average color of the line, the orientation of the line, or any suitable combination thereof. The neural network may also be configured to predict a segmentation mask of the line, for example, by predicting which pixels are line pixels. Similarly, if debris is to be simulated, then the neural network may be configured to predict a debris mask. Supervised training for one or more of these variables may be performed by adding a loss function for each variable implemented. In cases where the neural network machine is to be trained on a mixture of real and simulated images, auxiliary losses for the real image may be omitted within each training batch. In some example embodiments, extra supervision may allow the neural network machine to learn more efficiently from a limited amount of source data.

To accurately train the neural network machine to recognize extremely faint test result lines, it may be helpful to generate simulated images with test result lines having extremely low alpha values, and this process is likely to create some simulated images with test result lines that are do not get detected. It is unknown in advance which images will have undetected or undetectable test result lines. Therefore, it may be beneficial for the loss function to handle these borderline or otherwise difficult cases gracefully, such that these cases do not dominate the training. Accordingly, the gradient for the alpha loss may be configured to not be large for alphas that are very close to zero. For example, squared error may be a reasonable alpha loss, but not squared error of log(alpha), which would explode near zero. Additionally, it might not be helpful or worthwhile to perform supervised training for certain parameters that would be unknown for undetectable lines. Examples of such parameters include line location, line boundaries, line color, and line mask. The corresponding loss functions for such parameters may be turned off (e.g., by multiplying them by zero) whenever the ground-truth alpha is below some predetermined threshold value for alpha.

As noted above, simulated images of LFA test strips may be used to pre-train a neural network machine (e.g., pre-train an object detection model implemented by a neural network that is implemented by the neural network machine), and the pre-trained neural network machine may then be fine-tuned with further training on real images of LFA test strips. This two-phase training approach may be especially useful if the set of real images is very small or if the set of real images lacks variation in lighting, fluid gradients, debris, or any combination thereof. The fine-tuning phase may be performed with real images only (e.g., if aiming to have a small number of operations, a small learning rate, or both). Alternatively, the fine-tuning phase may be performed with a mixture of real and synthetic images, for example, with different loss functions being turned on and off for different types of images. If the set of real images is extremely small, then it may suffice to fine-tune only the top few layers of the neural network, to prevent overfitting. In an extreme case, a separate model may be trained to take the predicted alpha as its only feature relevant for predictions (e.g., qualitative, quantitative, or both) in real images. As noted above, the relationship between alpha and concentration may be mathematically modeled by an equation. Thus, the inverse of that equation or a neural network layer with non-linearity can be used to specify a model that predicts concentration from alpha. Such a specified model may be trained by itself or in conjunction with the top few layers of the net in an end-to-end manner.

If the target application is qualitative analysis of images depicting LFA test strips, then it may be helpful to set a threshold value for the predicted alpha value to determine whether a line is present or not present. This threshold value can be determined by collecting a calibration set of images depicting real or simulated LFA test strips, plotting the receiver operating characteristic (ROC) curve, and choosing the best trade-off between true positive results and false positive results, based on the specific goals of the application. Ideally, the calibration set of images is separate from the testing set of images used for clinical validation, as the threshold value for alpha has been optimized against this particular calibration set.

If the target application is a quantitative analysis of images depicting LFA test strips, then it may be helpful to train the neural network to directly determine the analyte concentration. However, the gradient descent may work best if the concentration is standardized to have a mean of 0 and standard deviation of 1. If the concentration is exponentially distributed, then it may help to train the neural network to predict the log concentration, since the log concentration will be uniformly distributed. As a result, the training will not be dominated by high-concentration examples. Overall, it may be beneficial to stack a few more layers of non-linearity over the alpha predictions and then train the neural network end-to-end to predict the concentration.

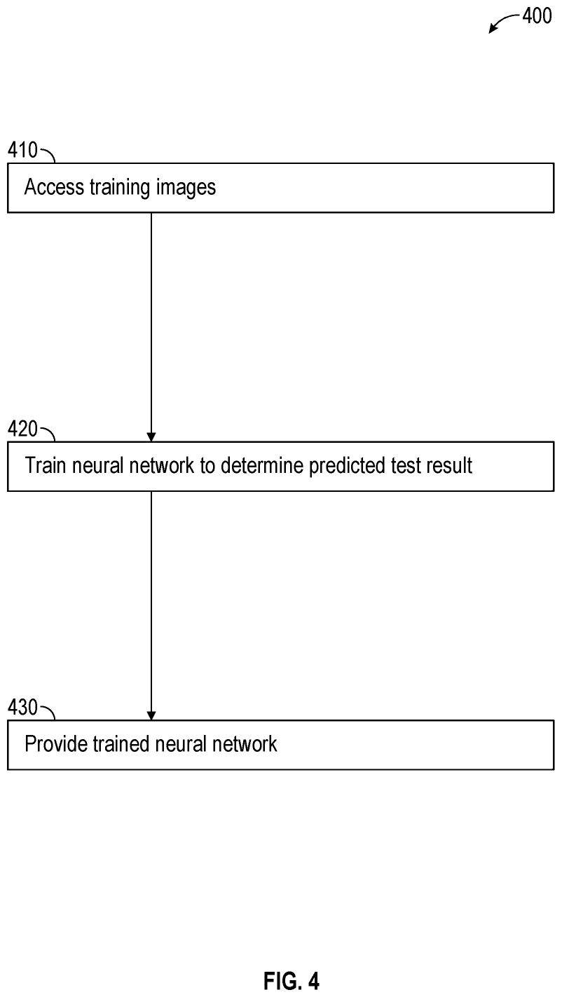

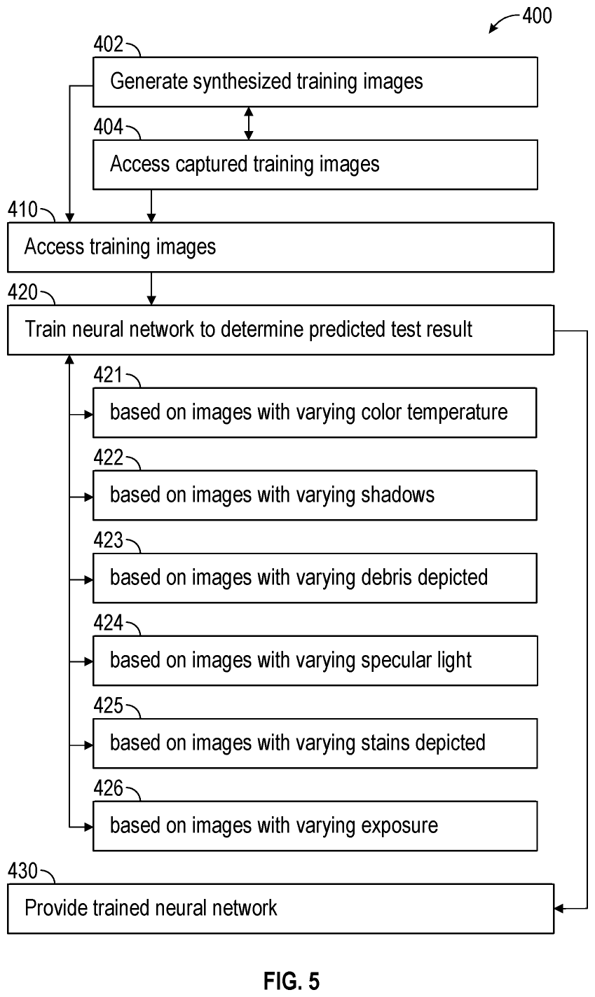

FIGS. 4 and 5 are flow charts illustrating a method 400 of training a neural network analysis to analyze LFA test strips, according to some example embodiments. Operations in the method 400 may be performed by a machine (e.g., a cloud or other system of one or more server computers) and result in provision of a suitably trained neural network to a device (e.g., a smartphone). Accordingly, the operations in the method 400 may be performed using computer components (e.g., hardware modules, software modules, or any combination thereof), using one or more processors (e.g., microprocessors or other hardware processors), or using any suitable combination thereof. As shown in FIG. 4, the method 400 includes operations 410, 420, and 430, and as shown in FIG. 5, the method 400 may additionally include one or more of operations 402, 404, 421, 422, 423, 424, 425, and 426.

In operation 402, the machine that performs the method 400 generates synthesized training images (e.g., as first portion of training images) that depict (e.g., show) simulated test strips under simulated imaging conditions.

In operation 404, the machine that performs the method 400 accesses captured training images (e.g., as second portion of training images) that depict (e.g., show) real test strips under real imaging conditions. The accessing of some or all of these captured training images may include capturing such training images using one or more cameras, accessing a database or other repository of such training images, or any suitable combination thereof.

In operation 410, the machine that performs the method 400 accesses training images for training the neural network that will be provided (e.g., to a device) in operation 430. The accessed training images may include some of all of the output of, or other results from, performing one or both of operations 402 and 404.

In operation 420, the machine that performs the method 400 trains the neural network that will be provided (e.g., to a device) in operation 430. For example, the training of the neural network may be performed based on the training images accessed in operation 410. Accordingly, based on the accessed training images, performance of operation 420 trains the neural network to determine a predicted test result based on an unlabeled image.

As shown in FIG. 5, one or more of operations 421, 422, 423, 424, 425, and 426 may be performed as part (e.g., a precursor task, a subroutine, or a portion) of operation 420, in which the neural network is trained.

In operation 421, the machine that performs the method 400 trains the neural network based on images that vary in color temperature (e.g., 1000K, 2000K, 2500K, 3000K, 3500K, 4000K, 5000K, 5200K, 6000K, 6500K, 7000K, 8000K, 9000K, 10,000K, or any suitable combination thereof).

In operation 422, the machine that performs the method 400 trains the neural network based on images that vary in their depiction of shadows (e.g., more shadows, fewer shadows, darker shadows, lighter shadows, locations of shadows, directions in which shadows fall, or any suitable combination thereof).

In operation 423, the machine that performs the method 400 trains the neural network based on images that vary in their depiction of debris (e.g., more debris, less debris, darker debris, lighter debris, locations of debris, color of debris, or any suitable combination thereof.

In operation 424, the machine that performs the method 400 trains the neural network based on images that vary in their depiction of specular light (e.g., more specular highlights, fewer specular highlights, size of specular highlights, locations of specular highlights, color of specular highlights, or any suitable combination thereof).

In operation 425, the machine that performs the method 400 trains the neural network based on images that vary in their depiction of stains (e.g., more stains, fewer stains, darker stains, lighter stains, locations of stains, color of stains, or any suitable combination thereof).

In operation 426, the machine that performs the method 400 trains the neural network based on images that vary in their exposure (e.g., more exposure or less exposure, as indicated by brightness, contrast, peak white level, black level, gamma curve, or any suitable combination thereof).

In operation 430, the machine that performs the method 400 provides the trained neural network (e.g., to a device, such as a smartphone) for use as described herein for analyzing LFA test strips.

According to various example embodiments, one or more of the methodologies described herein may facilitate automated analysis of test strips by a neural network (e.g., implemented in a neural network machine). Moreover, one or more of the methodologies described herein may facilitate generation of synthesized images depicting simulated test strips or portions thereof. Hence, one or more of the methodologies described herein may facilitate training of such a neural network, as well as improved performance of such trained neural network in analyzing images of test strips, compared to capabilities of pre-existing systems and methods.

When these effects are considered in aggregate, one or more of the methodologies described herein may obviate a need for certain efforts or resources that otherwise would be involved in automated analysis of test strips by a neural network. Efforts expended by a user in obtaining automated analysis of test strips may be reduced by use of (e.g., reliance upon) a special-purpose machine that implements one or more of the methodologies described herein. Computing resources used by one or more systems or machines (e.g., within a network environment) may similarly be reduced (e.g., compared to systems or machines that lack the structures discussed herein or are otherwise unable to perform the functions discussed herein). Examples of such computing resources include processor cycles, network traffic, computational capacity, main memory usage, graphics rendering capacity, graphics memory usage, data storage capacity, power consumption, and cooling capacity.

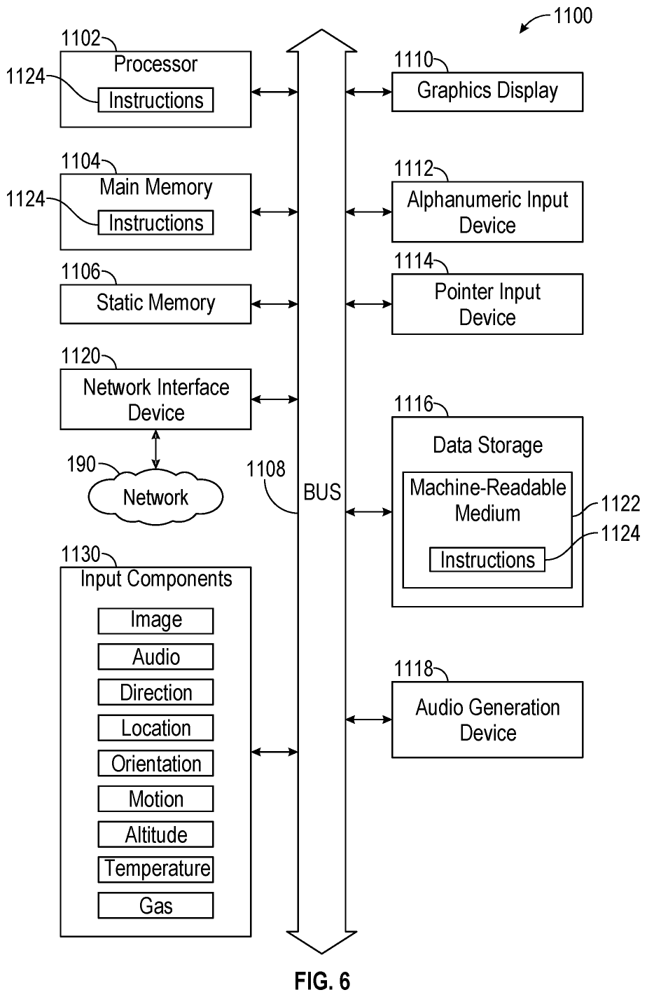

FIG. 6 is a block diagram illustrating components of a machine 1100, according to some example embodiments, able to read instructions 1124 from a machine-readable medium 1122 (e.g., a non-transitory machine-readable medium, a machine-readable storage medium, a computer-readable storage medium, or any suitable combination thereof) and perform any one or more of the methodologies discussed herein, in whole or in part. Specifically, FIG. 6 shows the machine 1100 in the example form of a computer system (e.g., a computer) within which the instructions 1124 (e.g., software, a program, an application, an applet, an app, or other executable code) for causing the machine 1100 to perform any one or more of the methodologies discussed herein may be executed, in whole or in part.

In alternative embodiments, the machine 1100 operates as a standalone device or may be communicatively coupled (e.g., networked) to other machines. In a networked deployment, the machine 1100 may operate in the capacity of a server machine or a client machine in a server-client network environment, or as a peer machine in a distributed (e.g., peer-to-peer) network environment. The machine 1100 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 1124, sequentially or otherwise, that specify actions to be taken by that machine. Further, while only a single machine is illustrated, the term “machine” shall also be taken to include any collection of machines that individually or jointly execute the instructions 1124 to perform all or part of any one or more of the methodologies discussed herein.

The machine 1100 includes a processor 1102 (e.g., one or more central processing units (CPUs), one or more graphics processing units (GPUs), one or more digital signal processors (DSPs), one or more application specific integrated circuits (ASICs), one or more radio-frequency integrated circuits (RFICs), or any suitable combination thereof), a main memory 1104, and a static memory 1106, which are configured to communicate with each other via a bus 1108. The processor 1102 contains solid-state digital microcircuits (e.g., electronic, optical, or both) that are configurable, temporarily or permanently, by some or all of the instructions 1124 such that the processor 1102 is configurable to perform any one or more of the methodologies described herein, in whole or in part. For example, a set of one or more microcircuits of the processor 1102 may be configurable to execute one or more modules (e.g., software modules) described herein. In some example embodiments, the processor 1102 is a multicore CPU (e.g., a dual-core CPU, a quad-core CPU, an 8-core CPU, a 128-core CPU, or any suitable combination thereof) within which each of multiple cores behaves as a separate processor that is able to perform any one or more of the methodologies discussed herein, in whole or in part. Although the beneficial effects described herein may be provided by the machine 1100 with at least the processor 1102, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 1100 may further include a graphics display 1110 (e.g., a plasma display panel (PDP), a light emitting diode (LED) display, a liquid crystal display (LCD), a projector, a cathode ray tube (CRT), or any other display capable of displaying graphics or video). The machine 1100 may also include an alphanumeric input device 1112 (e.g., a keyboard or keypad), a pointer input device 1114 (e.g., a mouse, a touchpad, a touchscreen, a trackball, a joystick, a stylus, a motion sensor, an eye tracking device, a data glove, or other pointing instrument), a data storage 1116, an audio generation device 1118 (e.g., a sound card, an amplifier, a speaker, a headphone jack, or any suitable combination thereof), and a network interface device 1120.

The data storage 1116 (e.g., a data storage device) includes the machine-readable medium 1122 (e.g., a tangible and non-transitory machine-readable storage medium) on which are stored the instructions 1124 embodying any one or more of the methodologies or functions described herein. The instructions 1124 may also reside, completely or at least partially, within the main memory 1104, within the static memory 1106, within the processor 1102 (e.g., within the processor's cache memory), or any suitable combination thereof, before or during execution thereof by the machine 1100. Accordingly, the main memory 1104, the static memory 1106, and the processor 1102 may be considered machine-readable media (e.g., tangible and non-transitory machine-readable media). The instructions 1124 may be transmitted or received over a network 190 via the network interface device 1120. For example, the network interface device 1120 may communicate the instructions 1124 using any one or more transfer protocols (e.g., hypertext transfer protocol (HTTP)).

In some example embodiments, the machine 1100 may be a portable computing device (e.g., a smart phone, a tablet computer, or a wearable device) and may have one or more additional input components 1130 (e.g., sensors or gauges). Examples of such input components 1130 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component (e.g., a gas sensor). Input data gathered by any one or more of these input components 1130 may be accessible and available for use by any of the modules described herein (e.g., with suitable privacy notifications and protections, such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 1122 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 1124 for execution by the machine 1100, such that the instructions 1124, when executed by one or more processors of the machine 1100 (e.g., processor 1102), cause the machine 1100 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 1124 for execution by the machine 1100 can be communicated via a carrier medium (e.g., a machine-readable carrier medium). Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 1124).

Certain example embodiments are described herein as including modules. Modules may constitute software modules (e.g., code stored or otherwise embodied in a machine-readable medium or in a transmission medium), hardware modules, or any suitable combination thereof. A “hardware module” is a tangible (e.g., non-transitory) physical component (e.g., a set of one or more processors) capable of performing certain operations and may be configured or arranged in a certain physical manner. In various example embodiments, one or more computer systems or one or more hardware modules thereof may be configured by software (e.g., an application or portion thereof) as a hardware module that operates to perform operations described herein for that module.

In some example embodiments, a hardware module may be implemented mechanically, electronically, hydraulically, or any suitable combination thereof. For example, a hardware module may include dedicated circuitry or logic that is permanently configured to perform certain operations. A hardware module may be or include a special-purpose processor, such as a field programmable gate array (FPGA) or an ASIC. A hardware module may also include programmable logic or circuitry that is temporarily configured by software to perform certain operations. As an example, a hardware module may include software encompassed within a CPU or other programmable processor. It will be appreciated that the decision to implement a hardware module mechanically, hydraulically, in dedicated and permanently configured circuitry, or in temporarily configured circuitry (e.g., configured by software) may be driven by cost and time considerations.

Accordingly, the phrase “hardware module” should be understood to encompass a tangible entity that may be physically constructed, permanently configured (e.g., hardwired), or temporarily configured (e.g., programmed) to operate in a certain manner or to perform certain operations described herein. Furthermore, as used herein, the phrase “hardware-implemented module” refers to a hardware module. Considering example embodiments in which hardware modules are temporarily configured (e.g., programmed), each of the hardware modules need not be configured or instantiated at any one instance in time. For example, where a hardware module includes a CPU configured by software to become a special-purpose processor, the CPU may be configured as respectively different special-purpose processors (e.g., each included in a different hardware module) at different times. Software (e.g., a software module) may accordingly configure one or more processors, for example, to become or otherwise constitute a particular hardware module at one instance of time and to become or otherwise constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory (e.g., a memory device) to which it is communicatively coupled. A further hardware module may then, at a later time, access the memory to retrieve and process the stored output. Hardware modules may also initiate communications with input or output devices, and can operate on a resource (e.g., a collection of information from a computing resource).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In some example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “accessing,” “processing,” “detecting,” “computing,” “calculating,” “determining,” “generating,” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

In view of the disclosure above, various examples of methods, machine-readable media, and systems (e.g., machines, devices, or other apparatus) are set forth below. It should be noted that one or more features of an example, taken in isolation or combination, should be considered within the disclosure of this application.

A first example provides a method comprising:

accessing, by one or more processors of a machine, training images that each depict a corresponding test strip of a corresponding test device under a corresponding combination of imaging conditions, the training images being each labeled with a corresponding indicator of a corresponding test result shown by the corresponding test strip;
training, by the one or more processors of the machine, a neural network to determine a predicted test result based on an unlabeled image that depicts a further test strip of a further test device under a corresponding combination of imaging conditions, the training being based on the training images; and providing, by the one or more processors of the machine (e.g., directly or indirectly), the trained neural network to a further machine configured to access the unlabeled image that depicts the further test strip of the further test device under the corresponding combination of imaging conditions and obtain the predicted test result from the trained neural network by inputting the unlabeled image into the trained neural network.

A second example provides a method according to the first example, wherein:

the corresponding combination of imaging conditions for the further test strip of the further test device includes a color temperature of the unlabeled image; and the neural network is trained based on a subset of the accessed training images, the subset varying in color temperature.

A third example provides a method according to the first example or the second example, wherein:

the corresponding combination of imaging conditions for the further test strip of the further test device includes a shadow on the further test strip; and
the neural network is trained based a subset of the accessed training images, the subset varying in presence of shadows on the corresponding test strips.

A fourth example provides a method according to any of the first through third examples, wherein:

the corresponding combination of imaging conditions for the further test strip of the further test device includes debris on the further test strip; and
the neural network is trained based a subset of the accessed training images, the subset varying in presence of debris on the corresponding test strips.

A fifth example provides a method according to any of the first through fourth examples, wherein:

the corresponding combination of imaging conditions for the further test strip of the further test device includes specular light on the further test strip; and
the neural network is trained based a subset of the accessed training images, the subset varying in presence of specular light on the corresponding test strips.

A sixth example provides a method according to any of the first through fifth examples, wherein:

the corresponding combination of imaging conditions for the further test strip of the further test device includes a stain on the further test strip; and
the neural network is trained based a subset of the accessed training images, the subset varying in presence of stains on the corresponding test strips.

A seventh example provides a method according to any of the first through sixth examples, wherein:

the corresponding combination of imaging conditions for the further test strip of the further test device includes exposure (e.g., more exposure or less exposure, as indicated by brightness, contrast, peak white level, black level, gamma curve, or any suitable combination thereof) of the unlabeled image; and
the neural network is trained based on a subset of the accessed training images, the subset varying in exposure.

An eighth example provides a method according to any of the first through seventh examples, further comprising:

generating, by the one or more processors of the machine, a first portion of the training images by generating a first set of synthesized images that each depict a corresponding simulated test strip under a corresponding combination of simulated imaging conditions; and
accessing, by the one or more processors of the machine, a second portion of the training images by accessing a second set of captured images that each depict a corresponding real test strip under a corresponding combination of real imaging conditions.

A ninth example provides a system (e.g., a computer system) comprising:

one or more processors; and
a memory storing instructions that, when executed by the one or more processors, configure the one or more processors to perform operations comprising:
accessing training images that each depict a corresponding test strip of a corresponding test device under a corresponding combination of imaging conditions, the training images being each labeled with a corresponding indicator of a corresponding test result shown by the corresponding test strip; training a neural network to determine a predicted test result based on an unlabeled image that depicts a further test strip of a further test device under a corresponding combination of imaging conditions, the training being based on the training images; and
providing the trained neural network to a further machine configured to access the unlabeled image that depicts the further test strip of the further test device under the corresponding combination of imaging conditions and obtain the predicted test result from the trained neural network by inputting the unlabeled image into the trained neural network.

A tenth example provides a system according to the ninth example, wherein:

the corresponding combination of imaging conditions for the further test strip of the further test device includes a color temperature of the unlabeled image; and
the neural network is trained based a subset of the accessed training images, the subset varying in color temperature.

An eleventh example provides a system according to the ninth example or the tenth example, wherein:

the corresponding combination of imaging conditions for the further test strip of the further test device includes a shadow on the further test strip; and
the neural network is trained based a subset of the accessed training images, the subset varying in presence of shadows on the corresponding test strips.

A twelfth example provides a system according to any of the ninth through eleventh examples, wherein:

the corresponding combination of imaging conditions for the further test strip of the further test device includes debris on the further test strip; and
the neural network is trained based a subset of the accessed training images, the subset varying in presence of debris on the corresponding test strips.

A thirteenth example provides a system according to the any of ninth through twelfth examples, wherein:

the corresponding combination of imaging conditions for the further test strip of the further test device includes specular light on the further test strip; and
the neural network is trained based a subset of the accessed training images, the subset varying in presence of specular light on the corresponding test strips.

A fourteenth example provides a system according to any of the ninth through thirteenth examples, wherein:

the corresponding combination of imaging conditions for the further test strip of the further test device includes a stain on the further test strip; and
the neural network is trained based a subset of the accessed training images, the subset varying in presence of stains on the corresponding test strips.

A fifteenth example provides a system according to any of the ninth through fourteenth examples, wherein:

the corresponding combination of imaging conditions for the further test strip of the further test device includes exposure (e.g., more exposure or less exposure, as indicated by brightness, contrast, peak white level, black level, gamma curve, or any suitable combination thereof) of the unlabeled image; and
the neural network is trained based on a subset of the accessed training images, the subset varying in exposure.

A sixteenth example provides a system according to any of the ninth through fifteenth examples, wherein the operations further comprise:

generating, by the one or more processors of the machine, a first portion of the training images by generating a first set of synthesized images that each depict a corresponding simulated test strip under a corresponding combination of simulated imaging conditions; and
accessing, by the one or more processors of the machine, a second portion of the training images by accessing a second set of captured images that each depict a corresponding real test strip under a corresponding combination of real imaging conditions.

A seventeenth example provides a machine-readable medium (e.g., a non-transitory machine-readable storage medium) comprising instructions that, when executed by one or more processors, configure the one or more processors to perform operations comprising:

accessing training images that each depict a corresponding test strip of a corresponding test device under a corresponding combination of imaging conditions, the training images being each labeled with a corresponding indicator of a corresponding test result shown by the corresponding test strip;

training a neural network to determine a predicted test result based on an unlabeled image that depicts a further test strip of a further test device under a corresponding combination of imaging conditions, the training being based on the training images; and
providing the trained neural network to a further machine configured to access the unlabeled image that depicts the further test strip of the further test device under the corresponding combination of imaging conditions and obtain the predicted test result from the trained neural network by inputting the unlabeled image into the trained neural network.

An eighteenth example provides a machine-readable medium according to the seventeenth example, wherein:

the corresponding combination of imaging conditions for the further test strip of the further test device includes a color temperature of the unlabeled image; and
the neural network is trained based a subset of the accessed training images, the subset varying in color temperature.

A nineteenth example provides a machine-readable medium according to the seventeenth example or the eighteenth example, wherein:

the corresponding combination of imaging conditions for the further test strip of the further test device includes a shadow on the further test strip; and
the neural network is trained based a subset of the accessed training images, the subset varying in presence of shadows on the corresponding test strips.

A twentieth example provides a machine-readable medium according to any of the seventeenth through nineteenth examples, wherein:

the corresponding combination of imaging conditions for the further test strip of the further test device includes debris on the further test strip; and
the neural network is trained based a subset of the accessed training images, the subset varying in presence of debris on the corresponding test strips.

A twenty-first example provides a machine-readable medium according to any of the seventeenth through twentieth examples, wherein:

the corresponding combination of imaging conditions for the further test strip of the further test device includes specular light on the further test strip; and
the neural network is trained based a subset of the accessed training images, the subset varying in presence of specular light on the corresponding test strips.

A twenty-second example provides a machine-readable medium according to any of the seventeenth through twenty-first examples, wherein:

the corresponding combination of imaging conditions for the further test strip of the further test device includes a stain on the further test strip; and
the neural network is trained based a subset of the accessed training images, the subset varying in presence of stains on the corresponding test strips.

A twenty-third example provides a machine-readable medium according to any of the seventeenth through twenty-second examples, wherein:

the corresponding combination of imaging conditions for the further test strip of the further test device includes exposure (e.g., more exposure or less exposure, as indicated by brightness, contrast, peak white level, black level, gamma curve, or any suitable combination thereof) of the unlabeled image; and
the neural network is trained based on a subset of the accessed training images, the subset varying in exposure.

A twenty-fourth example provides a machine-readable medium according to any of the seventeenth through twenty-third examples, wherein the operations further comprise:

generating, by the one or more processors of the machine, a first portion of the training images by generating a first set of synthesized images that each depict a corresponding simulated test strip under a corresponding combination of simulated imaging conditions; and

accessing, by the one or more processors of the machine, a second portion of the training images by accessing a second set of captured images that each depict a corresponding real test strip under a corresponding combination of real imaging conditions.

A twenty-fifth example provides a carrier medium carrying machine-readable instructions for controlling a machine to carry out the operations (e.g., method operations) performed in any one of the previously described examples.

What is claimed is:

1. A method comprising:
accessing, by one or more processors, training images that each depict a corresponding test strip of a corresponding test device under a corresponding combination of imaging conditions, the training images being each labeled with a corresponding indicator of a corresponding test result shown by the corresponding test strip;
training, by the one or more processors, a neural network to determine a predicted test result based on an unlabeled image that depicts a further test strip of a further test device under a corresponding combination of imaging conditions, the training being based on the training images; and
providing, by the one or more processors, the trained neural network to a further machine configured to access the unlabeled image that depicts the further test strip of the further test device under the corresponding combination of imaging conditions and obtain the predicted test result from the trained neural network by inputting the unlabeled image into the trained neural network.

2. The method of claim 1, wherein:
the corresponding combination of imaging conditions for the further test strip of the further test device includes a color temperature of the unlabeled image; and
the neural network is trained based on a subset of the accessed training images, the subset varying in color temperature.

3. The method of claim 1, wherein:
the corresponding combination of imaging conditions for the further test strip of the further test device includes a shadow on the further test strip; and
the neural network is trained based a subset of the accessed training images, the subset varying in presence of shadows on the corresponding test strips.

4. The method of claim 1, wherein:
the corresponding combination of imaging conditions for the further test strip of the further test device includes debris on the further test strip; and
the neural network is trained based a subset of the accessed training images, the subset varying in presence of debris on the corresponding test strips.

5. The method of claim 1, wherein:
the corresponding combination of imaging conditions for the further test strip of the further test device includes specular light on the further test strip; and
the neural network is trained based a subset of the accessed training images, the subset varying in presence of specular light on the corresponding test strips.

6. The method of claim 1, wherein:
the corresponding combination of imaging conditions for the further test strip of the further test device includes a stain on the further test strip; and
the neural network is trained based a subset of the accessed training images, the subset varying in presence of stains on the corresponding test strips.

7. The method of claim 1, wherein:
the corresponding combination of imaging conditions for the further test strip of the further test device includes exposure of the unlabeled image; and
the neural network is trained based on a subset of the accessed training images, the subset varying in exposure.

8. The method of claim 1, further comprising:
generating a first portion of the training images by generating a first set of synthesized images that each depict a corresponding simulated test strip under a corresponding combination of simulated imaging conditions; and
accessing a second portion of the training images by accessing a second set of captured images that each depict a corresponding real test strip under a corresponding combination of real imaging conditions.

9. A system comprising:
one or more processors; and
a memory storing instructions that, when executed by at least one processor among the one or more processors, cause the system to perform operations comprising:
accessing training images that each depict a corresponding test strip of a corresponding test device under a corresponding combination of imaging conditions, the training images being each labeled with a corresponding indicator of a corresponding test result shown by the corresponding test strip;
training a neural network to determine a predicted test result based on an unlabeled image that depicts a further test strip of a further test device under a corresponding combination of imaging conditions, the training being based on the training images; and
providing the trained neural network to a further machine configured to access the unlabeled image that depicts the further test strip of the further test device under the corresponding combination of imaging conditions and obtain the predicted test result from the trained neural network by inputting the unlabeled image into the trained neural network.

10. The system of claim 9, wherein:
the corresponding combination of imaging conditions for the further test strip of the further test device includes a color temperature of the unlabeled image; and
the neural network is trained based a subset of the accessed training images, the subset varying in color temperature.

11. The system of claim 9, wherein:
the corresponding combination of imaging conditions for the further test strip of the further test device includes a shadow on the further test strip; and
the neural network is trained based a subset of the accessed training images, the subset varying in presence of shadows on the corresponding test strips.

12. The system of claim 9, wherein:
the corresponding combination of imaging conditions for the further test strip of the further test device includes debris on the further test strip; and
the neural network is trained based a subset of the accessed training images, the subset varying in presence of debris on the corresponding test strips.

13. The system of claim 9, wherein:
the corresponding combination of imaging conditions for the further test strip of the further test device includes specular light on the further test strip; and
the neural network is trained based a subset of the accessed training images, the subset varying in presence of specular light on the corresponding test strips.

14. The system of claim 9, wherein:
the corresponding combination of imaging conditions for the further test strip of the further test device includes a stain on the further test strip; and
the neural network is trained based a subset of the accessed training images, the subset varying in presence of stains on the corresponding test strips.

15. The system of claim 9, wherein:
the corresponding combination of imaging conditions for the further test strip of the further test device includes exposure of the unlabeled image; and
the neural network is trained based on a subset of the accessed training images, the subset varying in exposure.

16. The system of claim 9, wherein the operations further comprise:
generating a first portion of the training images by generating a first set of synthesized images that each depict a corresponding simulated test strip under a corresponding combination of simulated imaging conditions; and
accessing a second portion of the training images by accessing a second set of captured images that each depict a corresponding real test strip under a corresponding combination of real imaging conditions.

17. A machine-readable medium comprising instructions that, when executed by one or more processors of a machine, cause the machine to perform operations comprising:
accessing training images that each depict a corresponding test strip of a corresponding test device under a corresponding combination of imaging conditions, the training images being each labeled with a corresponding indicator of a corresponding test result shown by the corresponding test strip;
training a neural network to determine a predicted test result based on an unlabeled image that depicts a further test strip of a further test device under a corresponding combination of imaging conditions, the training being based on the training images; and
providing the trained neural network to a further machine configured to access the unlabeled image that depicts the further test strip of the further test device under the corresponding combination of imaging conditions and obtain the predicted test result from the trained neural network by inputting the unlabeled image into the trained neural network.

18. The machine-readable medium of claim 17, wherein:
the corresponding combination of imaging conditions for the further test strip of the further test device includes a color temperature of the unlabeled image; and
the neural network is trained based a subset of the accessed training images, the subset varying in color temperature.

19. The machine-readable medium of claim 17, wherein:
the corresponding combination of imaging conditions for the further test strip of the further test device includes a shadow on the further test strip; and
the neural network is trained based a subset of the accessed training images, the subset varying in presence of shadows on the corresponding test strips.

20. The machine-readable medium of claim 17, wherein:
the corresponding combination of imaging conditions for the further test strip of the further test device includes debris on the further test strip; and
the neural network is trained based a subset of the accessed training images, the subset varying in presence of debris on the corresponding test strips.

21. The machine-readable medium of claim 17, wherein:
the corresponding combination of imaging conditions for the further test strip of the further test device includes specular light on the further test strip; and
the neural network is trained based a subset of the accessed training images, the subset varying in presence of specular light on the corresponding test strips.

22. The machine-readable medium of claim 17, wherein:
the corresponding combination of imaging conditions for the further test strip of the further test device includes a stain on the further test strip; and
the neural network is trained based a subset of the accessed training images, the subset varying in presence of stains on the corresponding test strips.

23. The machine-readable medium of claim 17, wherein:
the corresponding combination of imaging conditions for the further test strip of the further test device includes exposure of the unlabeled image; and
the neural network is trained based on a subset of the accessed training images, the subset varying in exposure.

24. The machine-readable medium of claim 17, wherein the operations further comprise:
generating a first portion of the training images by generating a first set of synthesized images that each depict a corresponding simulated test strip under a corresponding combination of simulated imaging conditions; and
accessing a second portion of the training images by accessing a second set of captured images that each depict a corresponding real test strip under a corresponding combination of real imaging conditions.
